(19) United States
(12) Patent Application Publication
(10) Pub. No.: US 2019/0015127 A1
(43) Pub. Date: Jan. 17, 2019

(54) BLADE POSITIONING SYSTEM

(71) Applicant: GYRUS ACMI, INC. D/B/A OLYMPUS SURGICAL TECHNOLOGIES AMERICA, Southborough,, MA (US)

(72) Inventors: Ming J. Cheng, W. Warwick, RI (US); David C. Church, Millington, TN (US); Moussa Sane, Collierville, TN (US); Riyad Moe, Madison, WI (US)

(21) Appl. No.: 15/745,206

(22) PCT Filed: Jun. 16, 2016
(86) PCT No.: PCT/US2016/037833

§ 371 Date: Jan. 16, 2018

Related U.S. Application Data
(60) Provisional application No. 62/222,477, filed on Sep. 23, 2015.

(51) Int. Cl. A61B 17/32; A61B 34/20

(52) U.S. Cl. CPC A61B 17/32002; A61B 2090/0811; A61B 34/20

(57) ABSTRACT

A medical instrument is disclosed that includes a body, and outer blade, and at least one sensing device for determining a rotational position of the outer blade, the inner blade, or both. The outer blade, the inner blade, or both are rotatably connected to the body. The sensing device comprises a sensor that generates an output signal corresponding to its position within a magnetic field generated by an image guidance system.

### FIELD

These teachings relate generally to medical instruments, and more particularly to medical instruments for use with image guidance systems.

### BACKGROUND

Image guidance systems can provide a surgeon with a virtual representation of one or more medical instruments relative to pre-operative and/or intra-operative images of the anatomy and/or a surgical site. More specifically, image guidance systems may advantageously allow a surgeon to guide one or more medical instruments through the anatomy, even though the medical, instruments and/or the surgical site may not be within the surgeon's direct line of sight.

In image-guided surgery, generally, one or more pre-operative and/or intra-operative images of the anatomy and/or of the surgical site are acquired via computerized axial tomography (CAT scan), magnetic resonance imaging (MRI), or the like. An image guidance system and/or a generator is provided on or near the patient, and its position relative to the patient is recorded and preferably remains constant. A sensing device is provided on one or more of the medical instruments. During use, in response to a magnetic field generated by the image guidance system and/or the generator, the sensing device generates one or more output signals representative of its location relative to the image guidance system, the generator, and/or the surgical site. The output signals are communicated to the image guidance system, which also maintains the pre-operative and/or intra-operative images of the anatomy and/or of the surgical site. The image-guidance system provides a virtual representation on a display of the position, location, and/or orientation of the one or more medical instruments relative to the pre-operative and/or intraoperative images. Thus, the surgeon can view, track, and/or guide the medical instruments relative to the surgical site, even though either or both may not be in the surgeon's line of sight.

As can be imagined, image guidance systems may be preferred in various procedures, especially in minimally invasive and hard-to-reach medical procedures, like some sinus procedures. There remains a need, however, to improve the medical instruments used with image guidance systems. For example, some attempts have been made to provide a debrider for use with image guidance systems. Generally, a debrider is a medical instrument that has an outer blade and an inner blade located within the outer blade. During a surgical procedure, one or both of the blades are rotated and, via a cutting feature on one or both of the blades, tissue, cartilage, bone, or the like can be shaved, cut, resected, abraded and/or removed. In order to use the debrider with an image guidance system, one or more sensing devices are typically provided near a distal end of the debrider, which may undesirably increase a section size of one or both of the blades. Moreover, some debriders are driven by an electric motor that produces magnetic fields, which can undesirably interfere with the sensing devices and/or the magnetic field generated by the image guidance system and/or the generator. Further, some debriders have electrosurgical capabilities that generate heat, which can undesirably interfere with sensing device functions when the sensing devices are located at or near a distal end of the debrider.

Accordingly, it may be desirable to provide a medical instrument for use with an image guidance system that overcomes at least one of the aforementioned challenges. For example, it may be attractive to have a method and/or a system for accurately determining the position of one or more blades of a medical instrument relative to each other, to the medical instrument, a surgical site, etc., while overcoming one of more of the aforementioned challenges. It may also be attractive to have a medical instrument that is part of a system that, can determine the position of the outer blade; can determine the rotational orientation of one or more of the cutting windows of the blades; or both. It may be desirable to have a debrider for use with an image guidance system where the position of the outer blade, including its rotational orientation relative to the body, the anatomy, a surgical site, etc., can be accurately determined while overcoming one of more of the aforementioned challenges.

Some examples of debriders and positioning devices can be found in U.S. Pat. Nos. 8,702,702, 8,670,816 and U.S. Patent Application Publication Nos. 2013/0225943 and 2012/0101370, all of which are incorporated by reference herein for all purposes.

### SUMMARY

The present teachings provide a medical instrument. The medical instrument may be a debrider. The medical instrument includes an inner blade and outer blade having an outer blade cutting window. The medical instrument includes a sensing device. In response to a magnetic field, the sensing device can provide an output signal indicative of the position of the sensor. The position of the outer blade, the outer blade cutting window, or both relative to the sensor is known or can be calculated and/or determined from the output signals. The output signal can be communicated to an image guidance system so that a virtual representation of the medical instrument, the position of the outer blade, the outer blade cutting window, or a combination thereof relative to one or more pre-operative and/or intra-operative images can be displayed. The virtual representation can include a rotational orientation of the outer blade cutting window so that its position relative to the anatomy or a surgical site can be determined.

The sensing device may include one or more sensors, one or more magnets, or a combination thereof. Preferably, the sensing device includes at least one sensor and at least one magnet. A magnetic field generated by the image guidance system, a generator, or both can induce Eddy currents in the one or more sensors and/or change the electrical current and/or a magnitude of the electrical current in the one or more sensors. Preferably, as the medical instrument is moved, the amount of induced Eddy currents change, the strength of the induced Eddy currents change, the electrical current changes, and/or the magnitude of the electrical current changes. The one or more output signals may be indicative of the induced Eddy currents and/or the electrical current. The image guidance system can determine and/or calculate the position and/or the orientation of the medical instrument, the position of the outer blade, or both from the one or more output signals.

The magnet also generates a magnetic field. The magnet can be attached to the medical instrument. Preferably, the magnet can be attached to the outer blade. The magnet can be attached to the outer blade near a proximal end thereof so that one or more of the challenges described in the background section of this disclosure can be overcome. As the outer blade is rotated, the magnet correspondingly rotates. As the outer blade and the magnet rotate, the magnetic field generated by the magnet can perturb the magnetic field generated by the image guidance system and/or the generator; can induce and/or change the induced Eddy currents in the one or more sensors; can change the electrical current in the sensors; or a combination thereof. The one or more output signals can also include information relating to or corresponding to the perturbed magnetic field generated by the image guidance system and/or the generator; the induced and/or changed induced Eddy currents in the one or more sensors; changes in the electrical current in the sensors; or a combination thereof. Using this information, the image guidance system can also calculate the rotational position and/or the orientation of the outer blade, the outer blade cutting window, or both.

One or more of the image guidance system, a sensor interface unit, a sensor control unit, or a combination thereof can amplify, digitize, compute, calculate, and/or interpret the one or more output signals, and display a representation of the medical instrument, the outer blade, the outer blade cutting window, or any other feature of the medical instrument on a display. The representation can be displayed over or in combination with one or more pre-operative and/or intra-operative images of the anatomy and/or a surgical site. The representation can be real-time. Accordingly, a surgeon can view and track one or more medical instruments relative to the anatomy and/or a surgical site, even though either or both may not be directly in the surgeon's line of sight. Advantageously, a surgeon, can view and track a rotational position or orientation of the outer blade cutting window relative to the anatomy and/or a surgical site.

The teachings herein provide a medical instrument comprising a body; a surgical element connected to the body, the surgical element is moveable relative to the body; a first sensor; and a magnet. The magnet perturbs a magnetic field generated by an image guidance device. In response to the magnetic field, the perturbed magnetic field, or both the first sensor generates and, communicates an output signal to the image guidance device so that a representation of the surgical element relative to the body, a site of interest in the anatomy, or both can be displayed on a display.

These teachings also provide a medical instrument that includes a body, and outer blade, and at least one sensing device for determining a rotational position of the outer blade. The outer blade is rotatably connected to the body. The sensing device comprises a sensor that generates an output signal corresponding to its position within a magnetic field generated by an image guidance system.

The teachings according to the disclosure include a method. The method includes a step of obtaining the medical instrument and the image guidance system according to the teachings herein. The method includes a step of moving the medical instrument within the magnetic field so that a magnitude of electric current in the sensor changes and/or Eddy currents in the sensor are induced. The method includes a step of displaying a representation of the outer blade, the medical instrument or both on, a display of the image guidance system based on the output signal.

### BRIEF DESCRIPTION OF THE DRAWINGS

FIG. 1 is a perspective view of the medical instrument.

FIG. 2 is a detailed perspective view of the cutting feature of FIG. 1.

FIG. 3 is a detailed perspective view of a portion of the medical instrument of FIG. 1.

FIG. 4 is detailed perspective view of a portion of the medical instrument of FIG. 1.

FIG. 5 is detailed perspective view of a portion of the medical instrument of FIG. 1.

FIG. 6 is detailed perspective view of a portion of the medical instrument of FIG. 1.

FIG. 7a is a perspective view of an outer blade for use with the medical instrument of FIG. 1.

FIG. 7b is a perspective view of an outer blade for use with the medical instrument of FIG. 1.

### DETAILED DESCRIPTION

The explanations and illustrations presented herein are intended to acquaint others skilled in the art with the teachings, its principles, and its practical application. Those skilled in the art may adapt and apply the teachings in its numerous forms, as may be best suited to the requirements of a particular use. Accordingly, the specific embodiments of the present teachings as set, forth are not intended as being exhaustive or limiting of the teachings. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

This disclosure claims priority to U.S. Provisional Application No. 62/222,477 filed on Sep. 23, 2015, the entire contents of which is hereby incorporated by reference herein for all purposes. The image guidance system can be any suitable system where one or more medical instruments and/or portions of one or more medical instruments can be viewed, tracked, monitored, and or guided in conjunction with one or more preoperative or intraoperative images. The image, guidance system may provide for a surgeon or user to view, track, and/or guide on a display one or more medical instruments relative to the anatomy and/or a surgical site, even though either or both may not be directly in the surgeon's line of sight. The image guidance system may be of the type or similar to the system disclosed in U.S. Patent Application Publication number U.S. 2013/0225943A1, the disclosure of which is hereby incorporated by reference herein for all purposes.

The medical instrument can be any medical instrument. Preferably, the medical instrument is suitable for use with any image guidance system. The medical instrument may be capable of being held and manually manipulated, or the medical instrument can be on a stand or other holder and computer controlled. The medical instrument may be a debrider or a microdebrider. The medical instrument may be of the type disclosed in commonly owned U.S. Pat. No. 8,920,419 B2, filed on Mar. 14, 2013, the disclosure of which is hereby incorporated by reference herein in its entirety. Preferably, the medical instrument includes one or more blades or tubes or, preferably, two or more blades or tubes. The medical instrument may be used to perform any suitable surgery. For example, the medical instrument can be used to perform a tonsillectomy, turbinoplasty, septoplasty, supraglottoplasty, sinus surgery, throat surgery, small joint arthroscopy, large joint arthroscopy, spinal surgery, disc surgery, the like, or a combination thereof.

The medical instrument can include one or more surgical elements. The surgical element can be any feature or combination of features for performing a surgical procedure. For example, the surgical element can be an interchangeable tip; a cutting window; an outer blade; an outer blade cutting window; an inner blade; an inner blade cutting window; an electrode; a cryogenic delivery port; etc. The surgical element can be located on any portion and/or at any location on the medical instrument. For example, the surgical element can be located on, at, or near the body; on, at, or near a proximal end of the medical instrument; on, at, or near a distal end of the medical instrument; or at any suitable location there between. Preferably, the surgical element is an interchangeable tip that extends from the body of the medical instrument.

The interchangeable tip may be used to perform a surgical procedure. The interchangeable tip may be securely or, preferably, removeably connected to the medical instrument, the body of the medical instrument, or both. The interchangeable tip may be replaced or changed depending on, the type of surgical procedure. For example, a larger tip or a smaller tip may be preferred depending on the specific type of procedure. Preferably, the interchangeable tip can be replaced before or after every surgical procedure.

The interchangeable tip may include wiring so that the interchangeable tip can be used with a monopolar energy source, a bi-polar energy source, free of energy, or a combination thereof. The interchangeable tip may be configured so that based upon the arrangement of the wires within the tip, the tip applies a monopolar energy, and/or a bipolar energy to a procedure site. Alternatively, the interchangeable tip may be devoid of wiring for use with a mechanical tip, thereby the tip may not be configured to apply either monopolar or bipolar energy to a surgical site.

The interchangeable tip may include an outer blade or tube and an inner blade or tube. One or both of the blades or tubes can rotate and/or be rotated. Preferably, during use, the inner blade is rotated and the outer blade is substantially stationary. Preferably, the inner blade is rotated with a motor and the outer blade is substantially stationary during use but can be rotated by rotating and/or otherwise manipulating a suitable feature on the medical instrument, such as a nosecone for example. The inner blade, the outer blade, or both may rotate or be rotated clockwise, counterclockwise, or both. The inner blade may rotate and/or move back and forth. The outer blade may be rotatable so that a cutting feature and/or an opening in the blade may be positioned and/or aligned to correspond to a surgical site. For example, during a procedure, a surgeon may rotate the outer blade and the corresponding blade opening so that the opening and/or the cutting feature aligns with a surgical site. Preferably, when the medical instrument is within the anatomy and/or out of the surgeon's line of sight, using the teachings disclosed herein a surgeon can adjust the outer blade and the corresponding blade opening or cutting feature without having to remove the medical instrument from the anatomy and/or adjust the blade within the anatomy via trial-and-error.

One or both of the blades may include one or more cutting features. The cutting feature may be any feature that may cut, resect, shave, and/or remove tissue cartilage, bone or the like. The cutting feature may be located anywhere on the medical instrument. Preferably, the cutting feature is located at or near a distal end of the medical instrument. The cutting feature may comprise one or more openings in the outer blade, the inner blade, or both. One or both of the openings may include sharp and/or serrated teeth, edges, or the like. Rotating one or both of the blades may cause the cutting feature to cut, resect, shave, and/or remove tissue cartilage, bone, or the like.

The medical instrument may include one or more sensing devices. The one or more sensing devices may cooperate with the image guidance system so that a surgeon can view, track, and/or guide one or more medical instruments through the anatomy even though the medical instrument and/or the anatomy may not be in the surgeon's line of sight. The one or more sensing devices may cooperate with the image guidance system to provide a relative location, position, and/or orientation of the medical instrument, the one or more blades, the interchangeable tip, the one or more cutting windows or cutting features, or a combination thereof relative to the medical instrument, the anatomy, a site of interest in the anatomy, or a combination thereof. Advantageously, the one or more sensing devices may cooperate with an image guidance system so that a surgeon can move or rotate the outer blade and the corresponding blade opening to align with a surgical site without having to remove the medical instrument from the anatomy to rotate the blade, for example.

The one or more sensing devices may comprise any suitable number and any suitable types of sensors, magnets, or both. For example, the one or more sensors may comprise or include any number of electromagnetic sensors, electromagnetic coils, digitizers, optical tracking sensors, visual sensors, IGS sensors, the like, or a combination thereof. The one or more sensors may sense one or more degrees of freedom, two or more degrees of freedom, three or more degrees of freedom, four or more degrees of freedom, or even five or more degrees of freedom (e.g., six degrees of freedom or more). The one or more magnets may be permanent magnets, electromagnets, or both.

The one or mote sensors, magnets, or both may be provided or attached to any portion of the medical instrument. Preferably, the one or more sensors, magnets, or both are provided or attached at a location on the medical instrument and/or the interchangeable tip where the sensors do not contact the anatomy; do not increase a section size of the medical instrument, the one or more blades, or a combination thereof; are at a reduced risk of shorting or being heated; or a combination thereof. In some configurations, the one or more sensors, magnets, or both may be provided on the body of the medical, instrument, the nosecone, or both and separate from the interchangeable tip. In this regard, advantageously, the one or more sensors, magnets, or both do not have to be discarded after an interchangeable tip is set aside, replaced, or discarded. This may reduce waste and/or the cost of the one or more interchangeable tips, in some configurations, the one or more sensors, magnets, or both may be provided on the interchangeable tip, the inner blade, the outer blade, and on one or more mechanisms or members. The one or more mechanisms or members may be used to move, relocate, and/or reposition the interchangeable tip and/or one or more of the surgical elements relative to the medical instrument, the body, the handle, a surgical site or environment, or a combination thereof. For example, the one or more sensors, magnets, or both may be located may be located on a first member, a second member, or both. In some configurations, it may be advantageous if the one or more sensors, magnets, or both are attached to the tip, the inner blade, the outer blade, the one or more mechanisms, members, or a combination thereof so that the one or more sensors, magnets or both to not move relative to one another. Preferably, the one or more sensors, magnets, or both may be located near a proximal end of the interchangeable tip, such as where the interchangeable tip contacts the body of the medical instrument. Preferably, the one or more sensors, magnets, or both are provided on the medical instrument and their location or position relative to a surgical element, a distal end of the interchangeable tip, the one or more cutting windows, the outer blade cutting window, or a combination thereof is known and remains fixed.

Preferably, during use, a magnetic field generated by the image guidance system induces Eddy currents in the one or more sensors; changes an electrical current and/or a magnitude of electrical current in the one or more sensors; or a combination thereof. Information relating to or corresponding to the one or more of the aforementioned effects of the magnetic field on the one or more sensors can be communicated to the image guidance system via one or more output signals. Alternatively, or in addition, the one or more sensors may perturb a magnetic field generated by the image guidance system, which can be communicated to the image guidance system in the same or via different output signals. From the one or more output signals, the image guidance system can calculate and/or determine the location and/or position of the distal end of the medical instrument, the interchangeable tip, the one or more cutting windows, the outer blade cutting window, or a combination thereof. More specifically, because the location of the one or more sensors relative to the location and/or position of the distal end of the medical instrument, the one or more cutting features, the outer blade cutting window, the surgical element, or a combination thereof is known, once the location of the sensor is known, the location and/or position of the distal end of the medical instrument, the one or more cutting features, the outer blade cutting window, the surgical element, or a combination thereof can be calculated and/or determined.

The one or more magnets may function to provide a relative location, position, and/or orientation of the medical instrument, the one or more blades, the interchangeable tip, the one or more windows, surgical elements, or a combination thereof. Preferably, the one or more magnets cooperate with the image guidance system, the one or more sensors, or both to provide a rotational position or location of the interchangeable tip, a distal end of the outer blade, the outer blade cutting window, the surgical element, or a combination thereof. The one or more magnets may be provided or attached to any portion of the medical instrument. Preferably, the one or more magnets are provided or attached at a location on the medical instrument where the magnets do not contact the anatomy; do not increase a section size of the medical instrument, the one or more blades, or a combination thereof, are at a reduced risk of shorting or heating; or a combination thereof. Preferably, the one or more sensors are provided on the outer blade, the inner blade, or both; on the body or on another suitable portion of the medical instrument; on one or more mechanisms used to move and/or manipulate the interchangeable tip; or a combination thereof. The one or more mechanisms may be used to move or manipulate the interchangeable tip relative to the medical instrument, the body, the handle, a surgical site or environment, or a combination thereof.

The one or more magnets may induce Eddy currents in the one or more sensors, which can be communicated to the image guidance system. The one or more magnets may perturb a magnetic field generated by the image guidance system, which can be communicated to the image guidance system. Information relating to or corresponding to the output signal, the induced eddy currents, the perturbed magnetic field, or a combination thereof can be communicated to the image guidance system via the one or more output signals from the sensors, via different signals, or any other suitable signal disclosed herein.

FIG. 1 illustrates a medical instrument 10. The medical instrument 10 includes a body 12 and a surgical element 14. The surgical element 14 can be an interchangeable tip 15. The interchangeable tip 15 includes an outer blade 16 and an inner blade 18 located within the outer blade 16. The body 12 includes a nosecone 20 for rotating the outer blade 16, and a motor 22 that rotates the inner blade 18. The body 12 also includes a connection enclosure 24. The interchangeable tip 15 includes a cutting feature 26 located at or near a distal end of the medical instrument 10.

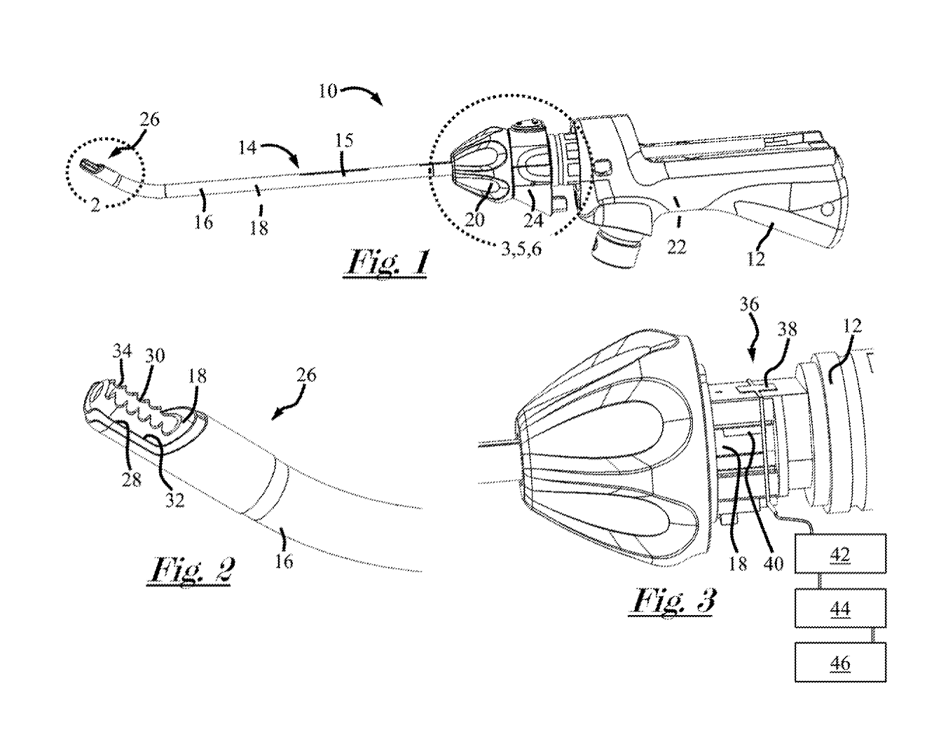

FIG. 2 is a detailed view of the cutting feature 26. The cutting feature 26 includes an outer blade cutting window 28 in the outer blade 16, and an inner blade cutting window 30 in the inner blade 18. The outer blade cutting window 28 includes outer window cutting edges 32, and the inner blade cutting window 30 includes inner window cutting edges 34.

FIG. 3 is a detailed view of a portion of the medical instrument 10 of FIG. 1 with the connection enclosure 24 removed for clarity. The medical instrument 10 includes a sensing device 36. The sensing device 36 includes a sensor 38 attached to a portion of the body 12 of the medical instrument 10, and a magnet 40 attached to the outer blade 16 of the interchangeable tip 15. The sensor 38 is in communication with a sensor interface unit 42, which is in communication with a system control unit 44, which is in communication with an image guidance system 46.

With reference to FIGS. 1-3, during use, the motor 22 rotates the inner blade 18 such that the cutting blade windows 28, 30 align with each other, as shown in FIG. 2, for example, and then become misaligned with each other (not shown). While rotating, tissue, cartilage, bone, and/or the like can be shaved, cut, resected, abraded and/or removed via the cutting blade windows 28, 30 and/or the window edges 30, 32.

The image guidance system 46 can generate a magnetic field. When the medical instrument 10 is in the magnetic field and then moved around, the magnetic field may induce Eddy currents in the sensor 38 and/or change the electrical current in the sensor 38. One or more output signals corresponding to the induced Eddy currents and/or the electrical current can be communicated via one or more output signals from the sensor 38 to the image guidance system 46. The image guidance system 46 can interpret the output signals and determine a position and/or location of the one or more sensors 38 within the magnetic field. Accordingly, because the position of the surgical element 14, the interchangeable tip 15, the outer blade 16, or a combination thereof relative to the sensor 38 is known and/or remains generally constant during use, the position and/or location of the surgical element 14, the interchangeable tip 15, the outer blade 16, or a combination thereof can be calculated and/or determined.

The nosecone 20 can be manipulated and/or rotated so that the outer blade 16 and/or the outer blade cutting window 28 rotates, orientates, and/or moves relative to the inner blade 18, the inner blade cutting window 30, the body 12, a site of interest in the anatomy, or a combination thereof. As the outer blade 16 is rotated, the magnet 40, which is attached to the outer blade 16, correspondingly rotates. As the magnet 40 rotates, a magnetic field generated by the magnet 40 induces Eddy currents in the sensor 38; perturbs the magnetic field generated by the image guidance system 46; and/or affects or changes a magnitude of the electrical current in the sensor 38, one or more of which may also be included in the one or more output signals provided by the sensor 38 to the image guidance system 46. Accordingly, therefore, based on the effects of the magnet 40 and/or the output signals, the image guidance system 46 can also determine a rotational position of the outer blade 16, the outer blade cutting window 28, or both relative to the inner blade 18, the inner blade cutting window 30, the medical instrument 10, the body 12, or a combination thereof.

Before the one or more output signals are communicated to the image guidance system 46, however, the output signals can be communicated to the sensor interface unit 42, which can amplify and digitize the output signals from the sensor. An output signal from the sensor interface unit 42 can be generated and provided to a system control unit 44. The system control unit 44 can analyze and/or compute the position and/or orientation of the surgical element 14, the interchangeable tip 15, the outer blade 16, the outer blade cutting window 28, or a combination thereof. An output from the system control unit 44 can be provided to the image guidance system 46. The image guidance system 46 maintains one or more pre-operative and/or intra-operative images of the anatomy and/or of the surgical site. The image guidance system 46 can display in real time the medical instrument 10, body 12, the outer blade 16, the outer blade cutting windows 30, and/or any other components of the medical instrument 10 relative to the anatomy, a surgical site, or a combination thereof acquired via the pre-operative and/or intra-operative images.

FIG. 4 is a detailed view of a portion of the medical instrument 10 of FIG. 1 with the connection enclosure 24 removed for clarity. The medical instrument 10 includes a sensing device 36. The sensing device 36 includes a sensor 38 attached to the outer blade 16 of the interchangeable tip 15. Accordingly, when the outer blade 16 is rotated, the sensor 38 correspondingly rotates. Preferably, the sensor 38 is a five degree of freedom sensor or a six degree of freedom sensor. The sensor 38 includes a plurality of leads or pogo pins, each in communication with a corresponding slip ring 50, 52, 54. Each slip ring 50, 52, 54 carries a corresponding signal including Vᵢₙ, Vₒᵤₜ, and power, for example. The slip rings 50, 52, 54 prevent wire twisting when the outer blade 16 and therefore the sensor 38 are rotated. The sensor 38 is in communication with a sensor interface unit 42, which is in communication with a system control unit 44, which is in communication with an image guidance system 46.

With reference to FIGS. 1-2 and 4, during use, the motor 22 rotates the inner blade 18. Moreover, the nosecone 20 can be rotated to rotate, orientate, and/or reposition the outer blade 16 and/or the outer blade cutting window 28 relative to the inner blade 18, the inner blade cutting window 30, the body 12, a site of interest in the anatomy, or a combination thereof.

The image guidance system 46 can generate a magnetic field. When the medical instrument 10 is in the magnetic field and then moved around within the magnetic field, the magnetic field may include Eddy currents in the sensor 38 and/or may change the electrical current in the sensor 38. One or more output signals from the sensor 38 corresponding to the included Eddy currents and/or the change in the electrical current are communicated to the image guidance system 46 so that the position and/or location of the sensor 38 can be determined. Because the position of the surgical element 14, the interchangeable tip 15, the outer blade 16, or a combination thereof relative to the sensor 38 is known and/or remains generally constant, the position and/or location of the surgical element 14, the interchangeable tip 15, and/or the outer blade 16, can be calculated and/or determined from the one or more output signals. Moreover, the output signal can also include information pertaining to or corresponding to a rotational position of the surgical element 14, the outer blade cutting window 28 relative to the inner blade 18, the inner blade cutting window 30, the medical instrument 10, the body 12, or a combination thereof.

Before the one or more output signals are communicated to the image guidance system 46, however, the output signals can be communicated to the sensor interface unit 42, which can amplify and digitize the output signals from the sensor. An output signal from the sensor interface unit 42 can be generated and provided to a system control unit 44. The system control unit 44 can analyze and/or compute the position and/or orientation of the surgical element 14, the interchangeable tip 15, the outer blade 16, the outer blade cutting window 28, or a combination thereof. An output from the system control unit 44 can be provided to the image guidance system 46. The image guidance system 46 also maintains one or more pre-operative and/or intra-operative images of the anatomy and/or of the surgical site. Accordingly, the image guidance system 46 can display in real time the medical instrument 10, body 12, the outer blade 16, the outer blade cutting windows 30 and/or any other components of the medical instrument 10 relative to the anatomy, a surgical site, or a combination thereof acquired via pre-operative and/or intra-operative images.

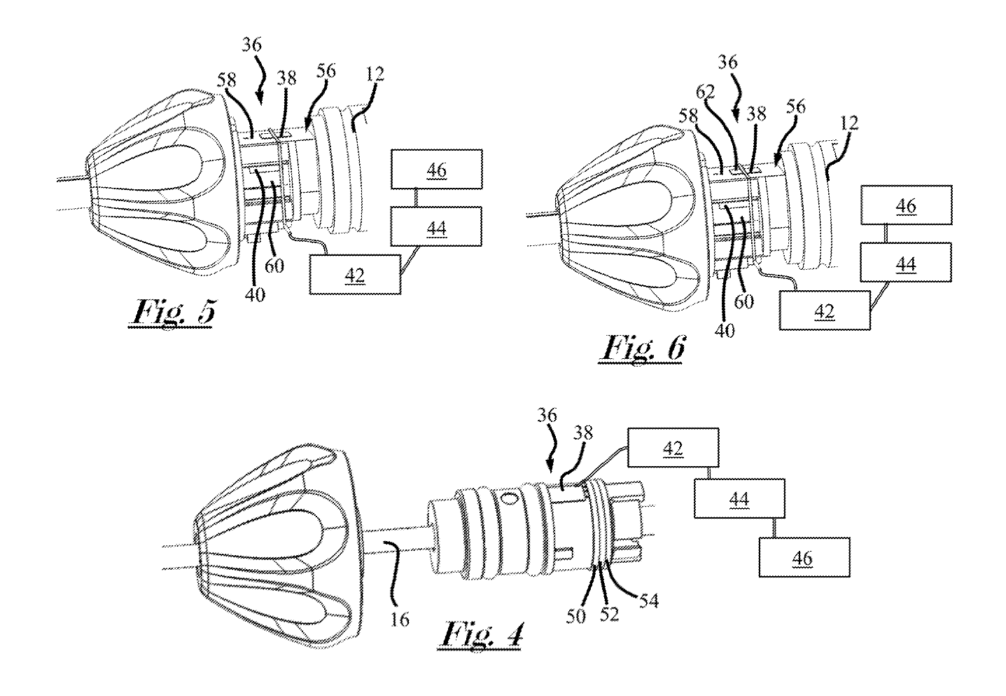

FIG. 5 is a detailed view of a portion of the medical instrument 10 of FIG. 1 with the connection enclosure 24 removed for clarity. The medical instrument 10 includes a mechanism 56 for moving, relocating, repositioning, and/or reorienting the interchangeable tip 14. The mechanism 26 includes a first member 58 and a second member 60.

The medical instrument 10 includes a sensing device 36. The sensing device 36 includes a sensor 38 attached to the first member 58 and a magnet 40 attached to the second member 60, or vice versa. The sensor 38 is in communication with a sensor interface unit 42, which is in communication with a system control unit 44, which is in communication with an image guidance system 46.

With reference to FIGS. 1-2 and 5, during use, the image guidance system 46 can generate a magnetic field. When the medical instrument 10 is in the magnetic field and/or moved around within the magnetic field, the magnetic field can induce Eddy currents in the sensor 38 and/or can change electrical current in the sensor 38, which may be included in one or more corresponding output signals communicated from the sensor 38 to the image guidance system 46. Accordingly, a position and/or location of the sensor 38 can be determined by the system 46 from the one or more output signals from the sensor 38. Because the position of the surgical element 14, the interchangeable tip 15, the outer blade 16, or a combination thereof relative to the sensor 38 is known and/or remains generally constant, once or after the position of the sensor 38 is known, the position and/or location of the surgical element 14, the tip 15, the outer blade 16, or a combination thereof can be calculated and/or determined.

When the interchangeable tip 14, the outer blade 16 and/or the outer blade cutting window 28 is moved, the second member 58 may correspondingly move. Accordingly, as the second member 58 moves, the magnet 40 correspondingly moves. As the magnet 40 moves, the magnetic field generated by the magnet 40 induces Eddy currents in the sensor 38; perturbs the magnetic field generated by the image guidance system 46; and/or changes the electrical current in the sensor 38. This information may be included in the one or more output signals provided by the sensor 38 to the image guidance system 46, or via any other suitable signal that may be disclosed herein. Accordingly, the image guidance system 46 can also determine movement in the second member 58, which may thus indicate movement in the mechanism 56, and, therefore, movement of the interchangeable tip 14, the outer blade cutting window 28, or both, for example.

Before the one or more output signals are communicated to the image guidance system 46, the output signals can be communicated to the sensor interface unit 42, which can amplify and digitize the output signals from the sensor. An output signal from the sensor interface unit 42 can be generated and provided to a system control unit 44. The system control unit 44 can analyze and/or compute the position and/or orientation of the surgical element 14, the interchangeable tip 15, the outer blade 16, the outer blade cutting window 28, or a combination thereof. An output from the system control unit 44 can be provided to the image guidance system 46. The image guidance system 46 also maintains one or more pre-operative and/or intra-operative images of the anatomy and/or of the surgical site. Accordingly, the image guidance system 46 can display in real time the medical instrument 10, body 12, the outer blade 16, the outer blade cutting windows 30 and/or any other components of the medical instrument 10 relative to the anatomy, a surgical site, or a combination thereof acquired via pre-operative and/or intra-operative images.

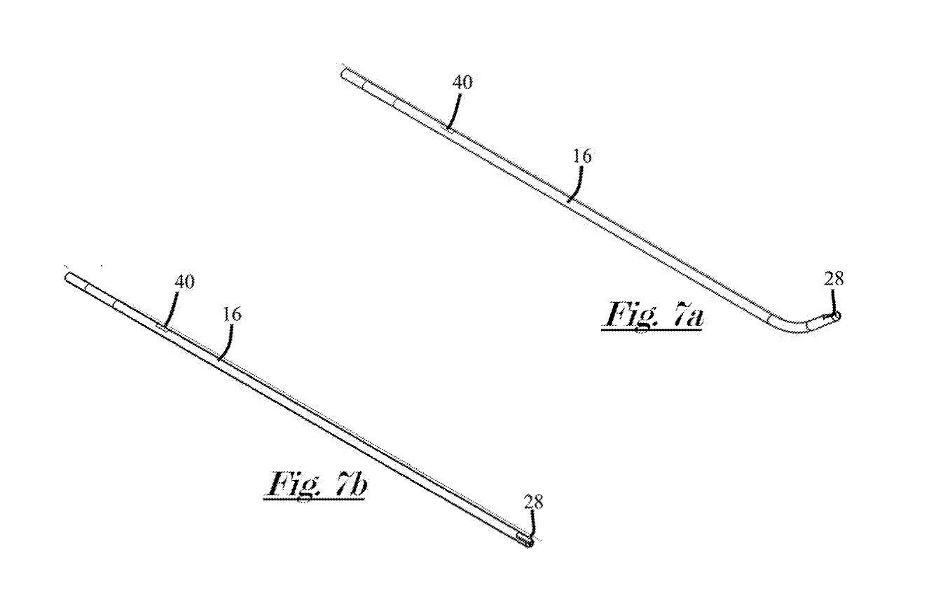

FIG. 6 is a detailed view of a portion of the medical instrument 10 of FIG. 1 with the connection enclosure 24 removed for clarity. The medical instrument 10 includes a mechanism 56 for moving, relocating, repositioning, and/or reorienting the interchangeable tip 14 relative to the body 12, a site of interest in the anatomy, or both. The mechanism 26 includes a first member 58 and a second member 60.

The medical instrument 10 includes a sensing device 38 including a first sensor 38 and a second sensor 62, both of which are attached to the first member 58. It may be desirable for the sensors 38, 62 to be attached to the first member 58 such that there is little to no movement, rotative movement, motion, or a combination thereof between the sensors 38, 62, relative to one another and independent of any movement of one or both of the members 58, 60. It is contemplated that the medical instrument 10 is capable of detecting relative position or orientation of the first member relative to the second member. The sensing device 38 also includes a magnet 40 attached to the second member 60. The sensors 38, 62 are in communication with a sensor interface unit 42, which is in communication with a system control unit 44, which is in communication with an image guidance system 46.

With reference to FIGS. 1-2 and 6, during use, the image guidance system 46 can generate a magnetic field. When the medical instrument 10 is in the magnetic field, the magnetic field can induce Eddy currents in one or both of the sensors 38, 62 and/or change the electrical current in one or both of the sensors 38, 62. The induced Eddy currents and/or the change in the electrical currents in one or both of the sensors 38, 62 can be communicated to the image guidance system 46 via one or more sensor output signals. A deviation between the output signals from the sensors 38, 62 can be indicative of their location and/or a change in a location of the sensors 38, 62 relative to one another. The deviation may correspond to relative motion of the mechanism 56 and thus movement (e.g., rotation) of the interchangeable tip 1 or the surgical element. Accordingly, the image guidance system 46 can determine a rotational position of the outer blade 16, the outer blade cutting window 28, or both relative to the inner blade 18, the inner blade cutting window 30, the medical instrument 10, the body 12, a surgical site, the anatomy, or a combination thereof.

Before the one or more output signals are communicated to the image guidance system 46, the output signals can be communicated to the sensor interface unit 42, which can amplify and digitize the output signals from the sensor. An output signal from the sensor interface unit 42 can be generated and provided to a system control unit 44. The system control unit 44 can analyze and/or compute the position and/or orientation of the surgical element 14, the interchangeable tip 15, the outer blade 16, the outer blade cutting window 28, or a combination thereof. An output from the system control unit 44 can be provided to the image guidance system 46. Accordingly, the image guidance system 46 can display in real time the medical instrument 10, body 12, the outer blade 16, the outer blade cutting windows 30 and/or any other components of the medical instrument 10 relative to the anatomy, a surgical site, or a combination thereof acquired via pre-operative and/or intra-operative images.

With continued reference to FIGS. 1-2 and 6, in another configuration, during use, the image guidance system 46 can generate a magnetic field. The magnet 40 can perturb the magnetic field generated by the image guidance system 46. In response to the perturbed magnetic field, the first sensor 38 (or the second sensor 62, or both) can send an output signal to the image guidance system 46. When the medical instrument 10 is within the magnetic field, the magnetic field can induce Eddy currents in one or both of the sensors 38, 62 and/or change the electrical current in the first sensor 38 (or the second sensor 62). The induced Eddy currents and/or the change in the electrical currents in the first sensor 38 (or the second sensor 62) can be communicated to the image guidance system 46 via one or more sensor output signals. The first and the second sensors 38, 62 can each send a baseline signal to the image guidance system 46 indicative of an initial position of the first member 58 relative to the second member 60. Once the medical instrument 10 and/or the interchangeable tip 14 and/or surgical element is moved relative to the body 12 within the magnetic field, the first and second sensors 38, 62 can each send additional signals to the image guidance system 46 corresponding to relative movement, relative position, or relative orientation of the first member 58 relative to the second member 60, or vice versa, as the tip 14 or surgical element is moved relative to the body 12. The image guidance system 46 can correspond a deviation between the baseline signals and the additional signals as relative motion in the mechanism (e.g., between the first and the second members 58, 60) so that a representation of the tip 14 or the surgical element relative to the body 12, a site of interest in an anatomy, or both can be displayed on a display.

With continued reference to FIGS. 1-2 and 6, in still yet another configuration, during use, the image guidance system 46 can generate a ma gait field. The magnet 40 can perturb the magnetic field. In response to the perturbed magnetic field, the first sensor 38 (or the second sensor 62) can send an output signal to the image guidance system 46. The first sensor 38 can generate and communicate a first sensor position output signal to the image guidance system 46 that corresponds to a position of the first sensor 38. Similarly, the second sensor 62 can generate and communicate a second sensor position output signal to the image guidance system 46 that corresponds to a position of the second sensor 62. When the medial instrument 10 and/or the surgical tip 14 is moved, in response to the perturbed magnetic field (i.e., via the magnet 40 and the field generated by the image guidance system 46), the first sensor position output signal may deviate from an initial position of the first sensor by a first sensor position error amount. Similarly, in response to the perturbed magnetic field, the second sensor position output signal may deviate from an initial position of the second sensor 62 by a second sensor position error amount. Preferably, the second sensor position error amount is less than the first sensor position error amount. More preferably, the second sensor position error amount is zero, which may be the case when the second sensor 62 is a sensor that is not affected by the magnetic field. For example, the second sensor 62 may be a visual sensor. The image guidance system 46 may determine a first sensor predicted position signal based on the second sensor position output signal, a second sensor predicted position signal based on the first sensor position output signal, or both, or a combination thereof. The image guidance system 46 can correspond a deviation between the first sensor predicted position signal and the first sensor position output signal as relative motion between the first member 58 and the second member 60 to determine and create a visual, real time position of the tip 14 or the surgical element on a display. Alternatively, or in addition, the image guidance system 46 can relate a deviation between the second sensor predicted position signal and the second sensor position output signal as relative motion between the first member 58 and the second member 60 to determine and create the visual, real time position of the tip 14 or the surgical element on the display.

FIGS. 7a and 7b each illustrate the outer blade 16. In FIG. 7a, the outer blade 16 is partially bent or angled between the proximal and distal ends. In FIG. 7b, the outer blade 16 is substantially straight. In both FIGS. 7a and 7b, the magnet 40 is generally aligned with the outer cutting blade window 28. The magnet 40 is attached to the outer blade 16 so that the position of the magnet 40 relative to the outer cutting blade window 28 does not change, even when the surgical element 14 moves and/or the outer blade 16 rotates.

Any numerical values recited, herein include all values from the lower value to the upper value in increments of one unit provided that there is a separation of at least 2 units between any lower value and any higher value. As an example, if it is stated that the amount of a component or a value of a process variable such as, for example, temperature, pressure, time and the like is, for example, from 1 to 90, preferably from 20 to 80, more preferably from 30 to 70, it is intended that values such as 15 to 85, 22 to 68, 43 to 51, 30 to 32 etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered, to be expressly stated in this application in a similar manner. As can be seen, the teaching of amounts expressed as “parts by weight” herein also contemplates the same ranges expressed in terms of percent by weight. Thus, an expression in the Detailed Description of the Teachings of a range in terms of at “‘x’ parts by weight of the resulting polymeric blend composition” also contemplates a teaching of ranges of same recited amount of “x” percent by weight of the resulting polymeric blend composition.”

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The disclosures of all articles and references, including patent applications and publications, are incorporated by reference for all purposes. The term “consisting essentially of” to describe a combination shall include the elements, ingredients, components, or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of the elements, ingredients, components or steps.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps.

It is understood that the above description is intended to be illustrative and not restrictive. Many embodiments as well as many applications besides the examples provided will be apparent to those of skill in the art upon reading the above description. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that, is disclosed herein is not a disclaimer of such subject matter, nor should it be regarded that the inventors did not consider such subject matter to be part of the disclosed inventive subject matter.

10 medical instrument

12 body

14 interchangeable tip

15 surgical element

16 outer blade

18 inner blade

20 nosecone

22 motor

24 connection enclosure

26 cutting feature

28 outer blade cutting window

30 inner blade cutting window

32 outer window cutting edges

34 inner window cutting edges

36 sensing device

38 sensor

40 magnet

42 sensor interface unit

44 system control unit

46 image guidance system

48 magnet

50 slip ring

52 slip ring

54 slip ring

56 mechanism

58 first member of mechanism 56

60 second member of mechanism 56

62 second sensor

1) A medical instrument comprising:
i. a body;
ii. a surgical element connected to the body, the surgical element being moveable relative to the body;
iii. a first sensor; and
iv. a magnet;
wherein the magnet perturbs a magnetic field generated by an image guidance system, and
wherein in response to the perturbed magnetic field, the first sensor generates and communicates an output signal to the image guidance system so that a representation of the surgical element relative to the body, a site of interest in an anatomy, or both is displayed on a display.

2) A medical instrument comprising:
i. a body;
ii. a surgical element connected to the body, the surgical element being moveable relative to the body;
iii. a mechanism for moving the surgical element relative to the body, the mechanism includes a first member and a second member;
iv. a first sensor attached to the first member;
v. a second sensor attached to the first member;
vi. a magnet attached to the second member;
wherein the magnet perturbs a magnetic field generated by an image guidance device,
wherein the first sensor generates and communicates a first sensor position output signal to the image guidance system, and the second sensor generates and communicates a second sensor position output signal to the image guidance system,
wherein in response to the perturbed magnetic field:
i. the first sensor position output signal deviates from an initial position of the first sensor by a first sensor position error amount, and
ii. the second sensor position output signal deviates from an initial position of the second sensor by a second sensor position error amount, the second sensor position error amount being less than the first sensor position amount,
wherein the image guidance system generates either;
i. a first sensor predicted position signal based on the second sensor position output signal, or
ii. a second sensor predicted position signal based on the first sensor position output signal, and
wherein the image guidance system corresponds either:
i. a deviation between the first sensor predicted position signal and the first sensor position output signal as relative motion between the first member and the second member to determine and create a visual, real time position of the surgical element on a display, or
ii. a deviation between the second sensor predicted position signal and the second sensor position output signal as relative motion between the first member and the second member to determine and create the visual, real time position of the surgical element on the display.

3) The medical instrument of claim 2, wherein the first sensor and the second sensor are attached to the first member such that there is no relative movement between the first sensor and the second sensor.

4) The medical instrument of claim 3, wherein one of the first sensor and the second sensor is an electromagnetic image guidance sensor, and
wherein the magnet affects the additional signal generated by the electromagnetic image guidance sensor when the first member, the second member, or both is moved, which causes the additional signals generated by the electromagnetic image guidance sensor to be different than the baseline signal generated by the electromagnetic image guidance sensor.

5) The medical instrument of claim 2, wherein at least one of the first sensor and the second sensor is an electromagnetic image guidance sensor.

6) The medical instrument according to claim 2, wherein the first sensor is an electromagnetic image guidance system sensor.

7) The medical instrument according to claim 2, wherein the first and the second sensors are an electromagnetic image guidance system sensor.

8) A medical instrument, comprising:
i. a body;
ii. an outer blade rotatably connected to the body; and
iii. at least one sensing device for determining a rotational position of the outer blade,
wherein the at least one sensing device comprises a sensor that generates an output signal corresponding to a position of the outer blade within a magnetic field generated by an image guidance system.

9) The medical instrument according to claim 8, wherein the outer blade includes an outer blade cutting window, and
wherein the at least one sensing device cooperates with the image guidance device to determine a rotational position of the outer blade cutting window relative to the body and/or a surgical site.

10) The medical instrument according to claim 8, wherein the magnetic field induces Eddy currents in the sensor, and information corresponding, to the induced Eddy currents is provided in the output signal.

11) The medical instrument according to claim 8, wherein the at least one sensing device comprises a magnet,
wherein the magnet perturbs the magnetic field and/or induces the Eddy currents in the sensor, and
wherein information corresponding to the induced Eddy currents and the perturbed magnetic field is provided in the output signal.

12) The medical instrument according to claim 11, wherein the magnet is provided on the outer blade and rotates with the outer blade when the outer blade is rotated, and
wherein the sensor is provided in the body.

13) The medical instrument according to claim 11, wherein the magnet is provided on the outer blade so that the magnet and the outer blade cutting window are generally aligned along a common plane.

14) The medical instrument according to claim 8, wherein the sensor is provided on the outer blade and rotates with the outer blade when the outer blade is rotated.

15) The medical instrument according to claim 8, wherein the sensor is a five degree of freedom sensor or a six degree of freedom sensor.

16) The medical instrument according to claim 8, wherein the sensor is in communication with at least one slip ring.

17) The medical instrument according to claim 8, wherein the output signal is communicated to a sensor interface unit that amplifies and digitizes the output signal.

18) The medical instrument according to claim 17, wherein an output from the sensor interface unit is communicated to a system control unit to generate a representation on a display of a rotational orientation of the outer blade, the outer blade cutting window, or both.

19) The medical instrument according to claim 8, wherein the outer blade is substantially straight between an outer blade proximal end and an outer blade distal end.

20) The medical instrument according to claim 8, wherein the outer blade is bent between an outer blade proximal end and an outer blade distal end.
